(19) United States
(12) Patent Application Publication
(10) Pub. No.: US 2025/0344941 A1
(43) Pub. Date: Nov. 13, 2025

(54) DEVICES, SYSTEMS, AND METHODS FOR DEFLECTABLE INSERTION DEVICES

(71) Applicant: Boston Scientific Scimed, Inc., Maple Grove, MN (US)

(72) Inventor: Richard C. TAH, Milford, MA (US)

(73) Assignee: Boston Scientific Scimed, Inc., Maple Grove, MN (US)

(21) Appl. No.: 19/203,621

(22) Filed: May 9, 2025

Related U.S. Application Data
(60) Provisional application No. 63/646,111, filed on May 13, 2024.

(51) Int. Cl. A61B 1/005; A61B 1/00; A61B 90/00

(52) U.S. Cl. CPC A61B 1/0052; A61B 1/00137; A61B 1/0055; A61B 1/0057; A61B 90/03; A61B 2090/034

(57) ABSTRACT

A medical device includes a shaft extending from a proximal end to a distal portion. The shaft includes a flexible section with at least one inwardly-projecting wall and an articulation section with multiple rotatable segments attached to the distal end of the flexible section. The medical device includes at least one control member that runs through the shaft, and at least one Bowden cable surrounding the control member in the flexible section. The Bowden cable terminates at an inwardly-projecting wall near the distal portion.

### CROSS-REFERENCE TO RELATED APPLICATIONS

This application claims the benefit of priority to U.S. Provisional Application No. 63/646,111, filed on May 13, 2024, which is incorporated by reference herein in its entirety.

### TECHNICAL FIELD

Aspects of the disclosure generally relate to devices, systems, and methods for deflectable insertion devices. Some aspects relate to medical devices, systems, and methods for interfacing or otherwise coupling an articulation section and a tube or other portion of a shaft of a medical device.

### BACKGROUND

During a medical procedure, a user may operate a medical device including a handle and a shaft extending distally from a portion of the handle. Some medical device shafts include a flexible section and an articulation section, which allows the user to deflect the distal portion of the medical device. The user may deflect the articulation section by actuating one or more pull wires or control members to direct the distal portion of the shaft, including the medical tools, toward an intended target. The control members may be tensioned or otherwise controlled by an actuating mechanism. Additionally, Bowden cables may radially surround the control members and facilitate the extension of the control members to the articulation section. The Bowden cable(s) help to transmit mechanical forces/energy through the movement of the pull wires.

However, in an exemplary construction of the shaft of the medical device, the flexible section of the shaft may be coupled to the articulation section with a coupler. In the exemplary construction, mechanisms to couple the Bowden cables to the coupler may require reduction of the outer diameter of the coupler in order for the coupler to fit into an interior space within the shaft. To maintain an adequate wall thickness for the coupler, the size of the inner space (e.g., the inner diameter) within a cavity of the coupler may be reduced. As a result, the reduced inner diameter may reduce the ability of the coupler and the shaft to receive medical tools (e.g., forceps, needles, brushes, etc.). In the construction of the assembly, it may also be necessary to further reinforce a joint between the flexible section, the coupler, and the articulation section with adhesive to help strengthen the joint(s), adding to the overall cost of the shaft.

The devices, systems, and methods of this disclosure may help to rectify one or more of the deficiencies described above or address other aspects of the art.

### SUMMARY

Examples of the disclosure relate to, among other things, coupling an articulation section and a flexible section of a shaft of the medical device, for example, without a separate coupler. Each of the examples disclosed herein may include one or more of the features described in connection with any other disclosed examples.

In an example, a medical device may include a shaft extending from a proximal end to a distal portion of the shaft. The distal portion of the shaft may include a flexible section. A distal portion of the flexible section may include at least one inwardly-projecting wall. The distal portion of the shaft may include an articulation section that may include a plurality of articulation segments coupled together and rotatable relative to each other. A proximal-most articulation segment may be attached to a distal end of the flexible section. In addition, the distal portion of the shaft may include at least one control member extending from the proximal end of the shaft to the distal end of the shaft and at least one Bowden cable surrounding at least one control member in the flexible section of the shaft. At least one Bowden cable may terminate at the at least one inwardly-projecting wall that may be proximate the distal portion of the flexible section.

Any examples described herein may have any of these features alone or in combination. The flexible section may include a tube that defines a plurality of slots. The tube may comprise a metal, a metal alloy, or a polymer. The distal portion of the flexible section that includes the at least one inwardly-projecting wall may not include the plurality of slots. The at least one inwardly-projecting wall may be an integral component of the tube and the at least one inwardly-projecting wall may be coupled to an inner surface of the tube. The at least one control member may be received within the inwardly-projecting wall. The at least one inwardly-projecting wall may be a perimeter of an aperture, and the at least one control member may extend through the aperture. The at least one control member may extend through an aperture defined by each articulation segment of the articulation section. Furthermore, the at least one control member may include two control members on opposite sides of the shaft, and the at least one Bowden cable may include two Bowden cables each surrounding one of the two control members. The flexible section may include two inwardly-projecting walls on opposite sides of an inner surface of the shaft. At least two control members and at least two Bowden cables may be positioned on opposite sides of the inner surface of the shaft. The medical device may also include a handle with an actuator operably coupled to the at least one control member, and when the actuator is manipulated the actuator tensions the at least one control member. Furthermore, the distal-most articulation segment of the plurality of articulation segments may be configured to be coupled to an end cap. The at least one control member may extend through the distal-most articulation segment and may be configured to terminate at the end cap.

In another example, a medical device may comprise a handle, a shaft, a plurality of pull wires, and a plurality of Bowden cables. The shaft may comprise a flexible area and an articulation area. The articulation area may be configured to releasably couple to a flexible tube area. A distal portion of the flexible tube area may include a plurality of curved segments. The plurality of pull wires may extend distally from the handle through the shaft. The plurality of pull wires may connect the flexible tube area and the articulation area. Furthermore, each of the plurality of Bowden cables may radially surround at least a portion of one of the plurality of pull wires. The plurality of Bowden cables may abut a proximal surface of the plurality of curved segments.

Any of the devices disclosed herein may include any of the following features in any combination. For example, each of the plurality of curved segments may be formed by an at least one inwardly projecting wall of the flexible tube area and form an aperture. The plurality of Bowden cables may abut a proximal face of an at least one inwardly-projecting wall. The plurality of pull wires may extend through an aperture defined by each articulation segment of the articulation area.

In another example, a medical device may comprise a shaft including a flexible section, an articulation section, at least one control member, and at least one Bowden cable. A distal portion of the flexible section may include at least one inwardly-projecting wall. The articulation section may include a plurality of articulation segments and at least one control member may extend from the proximal end of the shaft to the distal end of the shaft. A proximal-most articulation segment may be coupled to a distal end of the flexible section by at least one control member and at least one Bowden cable may surround at least a portion of at least one control member in the flexible section of the shaft. The at least one Bowden cable may terminate at the at least one inwardly-projecting wall.

### BRIEF DESCRIPTION OF THE FIGURES

The accompanying drawings, which are incorporated in and constitute a part of this application, illustrate exemplary aspects that, together with the written description, explain the principles of this disclosure. The figures depict exemplary aspects according to this disclosure, as follows:

FIG. 1 depicts a side-view of an exemplary medical device, including a handle and a shaft, according to some aspects of this disclosure.

FIG. 2A depicts a partial side view of an internal portion of a distal portion of the shaft of the medical device of FIG. 1.

FIG. 2B depicts a cross-section of FIG. 2A along the line A of the distal portion of the shaft.

FIG. 3A is a perspective partial view of the shaft of the medical device of FIG. 1.

FIG. 3B depicts a perspective end view of the distal end of the shaft of the medical device of FIG. 1

FIG. 3C depicts a cross-sectional view of the shaft of the medical device of FIG. 1.

### DETAILED DESCRIPTION

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements but may include other elements not expressly listed or inherent to such a process, method, article, or apparatus. In this disclosure, relative terms, such as, for example, “about,” “substantially,” “generally,” and “approximately” are used to indicate a possible variation of +10% in a stated value or characteristic.

Aspects of this disclosure are now described with reference to a medical device, systems, and methods for coupling an articulation section and a flexible section of a shaft of the medical device, for example, without a separate coupler. The medical device may include a handle and a shaft, including an articulation section and a flexible section. The medical device may also include a working channel extending the length of the shaft. The shaft may include at least one control member or pull wire extending from the proximal end of the handle to the distal end of the shaft. At least one control member may be positioned radially outward of the working channel within the shaft, and a Bowden cable may surround the at least one control member within the flexible section of the shaft. At least one Bowden cable may terminate at an inwardly-projecting wall proximate a distal end of the flexible section, while the control member extends distally to the articulation section. In these aspects, the at least one control member may connect the flexible tube section and the articulation section.

Reference will now be made in detail to examples to help illustrate aspects of the present disclosure through the accompanying drawings. Wherever possible, the same reference numbers will be used throughout the drawings to refer to the same or like parts.

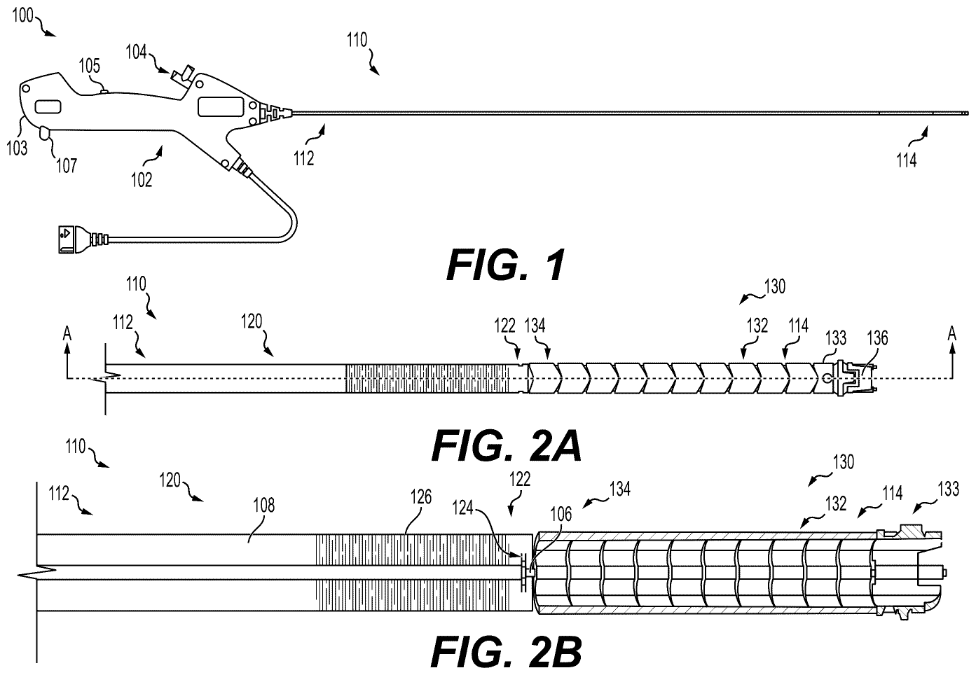

FIG. 1 illustrates an exemplary medical device 100, including a handle 102 and a shaft 110. The medical device 100 may be any suitable device configured to allow a user to access and view internal areas of a subject's body and perform medical diagnoses and/or treatments on a subject. For example, the medical device 100 may be an ureteroscope, an endoscope, a hysteroscope, a bronchoscope, a cystoscope, or other scopes or similar medical devices.

The shaft 110 may have a distal end 114. The distal end 114 may include one or more lighting elements or imaging devices. Distal end 114 of shaft 110 may be coupled to a cap, for example, including lighting elements or imaging devices and one or more lumens or distal openings (e.g., coupled to the working channel of the shaft). The lighting element(s) may include any element that may deliver light to a procedure site, such as light emitting diode(s), optical fibers, or other types of light sources. The imaging device(s) may include a camera and/or other optical device(s) for transmitting an image from a body lumen to a user (e.g., optical fiber, lens, image sensor, etc.). The distal end 114 may also include a distal opening (not shown) of a working channel of the shaft 110. As discussed in greater detail below, shaft 110 also includes a main section or a flexible section 120 and an articulation section 130, for example, distal of flexible section 120 (FIGS. 2A and 2B).

The working channel may extend from the handle, through the shaft, through the distal opening of the distal end 114. The handle 102 may include a port 104 in communication with the working channel. Auxiliary instruments, such as, for example, biopsy forceps, graspers, baskets, snares, and/or other devices may be inserted into the port 104 and advanced through the working channel to exit through the distal opening of the distal end 114 of the shaft 110 into a subject's body to perform a medical procedure. The shaft 110 may provide access into a bodily lumen (e.g., along a tortuous path, for example, via a natural body orifice).

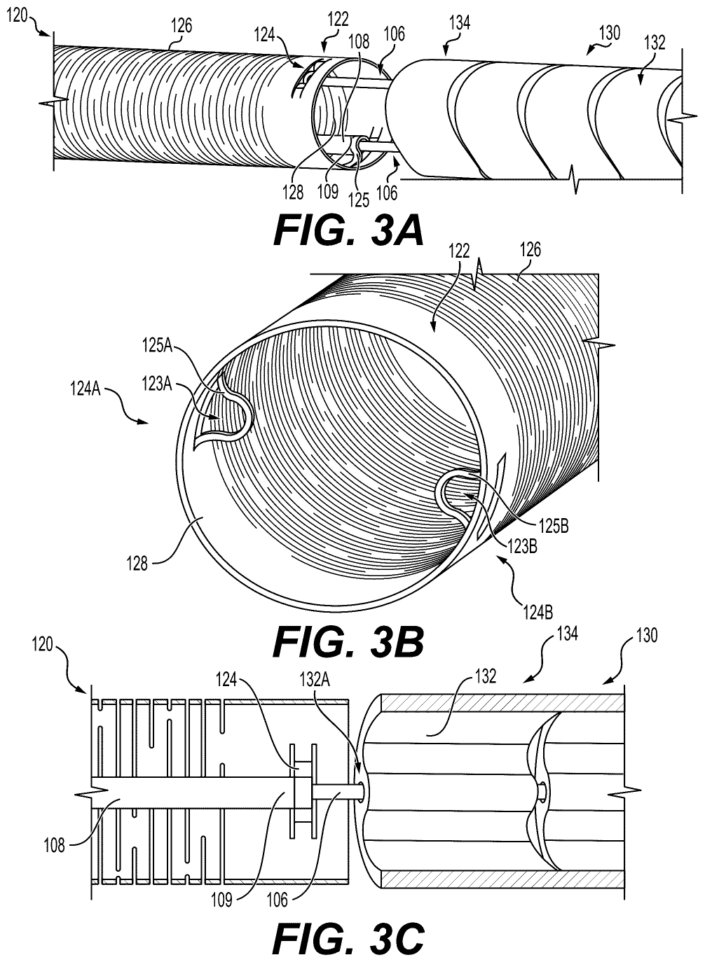

The handle 102 may also include one or more control elements for controlling aspects of the shaft 110. For example, the handle 102 may include one or more of the actuator 107 (e.g., knobs, thumbwheel, or levers). The actuator 107 may help steer the distal end 114 of the shaft 110 (e.g., by manipulating the articulation section 130 of the shaft 110). Additionally, the actuator 107 may be operably coupled (e.g., directly, or indirectly) to an articulation mechanism in the handle 102 and one or more control members 106 (e.g., pull wire(s)) extending from the proximal end 112 of the shaft 110 to the distal end 114, as shown in FIGS. 3A and 3C. The one or more of the image capture button 105 or other imaging control mechanism may control image/video capture, lighting levels, or one or more other aspects of the imaging device and/or the lighting elements.

As explained in more detail below, the shaft 110 includes a proximal end 112 and the distal end 114. The shaft 110 may be coupled to the distalmost end or distal portion of the handle 102. The proximal end 112 of the shaft 110 may include a flexible section 120 and the distal end 144 may include an articulation section 130. For example, the flexible section 120 of the shaft 110 may extend from the proximal end 112 along a majority of a length of the shaft 110 to a proximal end of the articulation section 130. The shaft 110 may include at least one control member 106 extending through the proximal end 112 of the shaft 110 to the distal end 114. As mentioned, the at least one control member 106 may extend proximally of the shaft 110, for example, to an internal coupling or control mechanism within the handle 102.

FIGS. 2A-2B depict side views of various internal portions of the shaft 110 of the medical device 100. It is noted that the shaft 110 may include one or more outer layers, sheaths, etc. to at least partially enclose the internal portions of the shaft 110, and that those outer layers, sheaths, etc. are omitted in FIGS. 2A-2B for illustration purposes. As discussed above, the shaft 110 includes the proximal end 112 and the distal end 114. The proximal end 112 of the shaft 110 may include the flexible section 120. The flexible section 120 may be composed of various materials, such as a metal, a metal alloy, a polymer, or other at least partially flexible material. The flexible section 120 may be a tube or tubular structure, a cylinder, or a suitable shape. For example, the flexible section 120 may be a tube defining a plurality of slots 126, for example, along a distal portion of the flexible section 120. The slots 126 may introduce areas or portions of flexibility along the length of the flexible section 120 of the shaft 110, allowing the shaft 110 to bend more easily in multiple directions and/or conform to the curvature of anatomical structures within a body lumen.

The slots 126 may be, for example, laser etched, cut, or otherwise formed on the flexible section 120. The slots 126 may extend through the thickness of the flexible section 120. The slots 126 may be evenly spaced along the length of the flexible section 120. Even spacing of the slots 126 along the length of the flexible section 120 may help to provide consistent flexibility along the length of the flexible section 120. Alternatively, the slots 126 may be concentrated in specific sections, such as the distal portion 122 of the flexible section 120. Concentrating the position of the slots 126 may be helpful for procedures requiring targeted flexibility. As discussed in more detail below, the distal portion 122 of the flexible section 120 does not include the slots 126. Rather, the distal portion 122 of the flexible section 120 may include at least an inwardly projecting wall 124.

The articulation section 130 may be constructed from materials suitable for providing a balance of flexibility and rigidity, such as metals, metal alloys, polymers, or other suitable materials. The articulation section 130 includes a plurality of articulation segments 132, for example, with adjacent articulation segments 132 coupled together. The articulation section 130 comprises multiple articulation segments 132, each of which may be capable of rotating relative to the adjacent segments 132. The articulation segments 132 may be coupled or connected together in a chain-like manner or otherwise form an at least partially flexible structure configured to adapt to the curvature of the anatomy. For example, a proximal-most articulation segment 134 may be attached to a distal-most end of the distal portion 122 of the flexible section 120. The articulation segments 132 may have cylindrical or segmented shapes that form a chain-like structure.

The shaft 110 of the medical device 100 may include or be coupled to an end cap 136. The end cap 136 may be affixed or otherwise coupled to the distal-most articulation segment 134. The end cap 136 may be releasable attached to the distal-most articulation segment 133. Additionally, the end cap 136 may feature design elements to facilitate its attachment to the articulation section 130 and the flexible section 120 of the shaft 110. For example, the end cap 136 may include one or more grooves, threads, or other mating features that allow for easy assembly and disassembly of the device.

The rotational movement of the articulation segments 132 may be controlled by the user via the actuator 107 discussed above. Each articulation segment 132 may be a distinct component of the articulation section 130. For example, including two or more articulation segments 132 of the articulation section 130. The actuator 107 may be operably coupled (e.g., directly, or indirectly) to one or more control members 106 (e.g., pull wire(s)) extending from the handle 102 and through the proximal end 112 of the shaft 110 to the distal end 114 and the articulation section 130. The user may engage or otherwise manipulate the actuator 107 to actuate the control member(s) 106 to deflect the articulation segments 132 or otherwise, rotate an articulation segment 132 relative to the adjacent segments 132.

Aspects of this disclosure are now described with reference to coupling the articulation section 130 and the flexible section 120 of the shaft 110 of the medical device 100. In an exemplary construction of the shaft 110 of the medical device 100 outside the scope of the disclosure, the flexible section 120 may be coupled to the articulation section 130 with a coupler, and the Bowden cable 108 may terminate at a proximal end of the coupler.

This disclosure introduces a system and method for directly interfacing the articulation section 130 with the flexible section 120, that is, without a coupler. The system and method include forming an inwardly projecting wall 124 in the distal portion 122 of the flexible section 120 that anchors the Bowden cable 108. The inwardly projecting wall 124 allows the control members 106 to pass through, but does not allow the Bowden cables 108 to pass through. Specifically, referring to FIG. 3B, distal portion 122 may include two inwardly projecting walls 124, forming respective apertures 123A and 123B. The control members 106 may pass through one of apertures 123A, 123B of the inwardly projecting walls 124 to connect to the flexible section 120 and the articulation section 130. The system may eliminate the need for a coupler and/or adhesive, which may help to simplify manufacturing, reduce costs, and/or provide one or more other benefits.

As discussed, the shaft 110 may include at least one control member 106 extending from the proximal end 103 of the handle 102, through the proximal end 112 of the shaft 110, and to the distal end 114 of the shaft 110. The at least one control member 106 may be radially outward of the working channel (not shown) of the shaft 110. Additionally, as shown in FIGS. 3A and 3C, a Bowden cable 108 may surround at least a portion of the at least one control member 106, for example, where the at least one control member 106 is within the flexible section 120 of the shaft 110. The at least one Bowden cable 108 terminates at a respective inwardly-projecting wall 124 proximate a distal portion 122 of the flexible section 120.

Furthermore, the control members 106 extends distally to the articulation section 130, which connects the flexible tube area or flexible section 120 and the articulation section 130. For example, the control member 106 may connect, couple, or attach the proximal-most articulation segment 133 to the distal portion 122 of the flexible section 120. The articulation section 130 may be releasably coupled to the flexible section 120. For example, the articulation section 130 may be disconnected from the flexible section 120 when the control members 106 are not extended to the articulation section 130.

In the embodiments contemplated herein, the Bowden cables 108 may not extend through apertures 123A, 123B (FIG. 3A, 3B). The Bowden cables 108 surround the control member 106 from the proximal end 103 of the handle 102 to the inwardly-projecting wall 124. Distal ends 109 of the Bowden cables 108 may abut a proximal surface or face of a respective one of the plurality of the curved segments 125A, 125B that form the inwardly-projecting walls 124 (e.g., FIG. 3B). The surface or face of the curved segments 125A, 125 may form the proximal faces of the inwardly-projecting walls 124. Furthermore, the diameter of the Bowden cables 108 may be greater than the outer diameter of the curved segments 125A, 125B. In other words, the Bowden cables 108 may contact, abut, or remain anchored to the curved segments 125A, 125B of the inwardly-projecting walls 124.

As shown in FIGS. 3A-3B, the distal portion 122 of the flexible section 120 may not include the slots 126, but instead includes at least one inwardly projecting wall 124. The inwardly projecting wall 124 may be an integral component of the tube of the flexible section 120. For example, the inwardly projecting wall 124 may be positioned on an inner surface 128 of the shaft 110 and the tube of the flexible section 120. The inwardly projecting wall 124 may be formed by crimping the distal portion 122 of flexible section 120. The crimping or other formation process may form the desired configuration of the at least one inwardly projecting wall 124. For example, the inwardly projecting wall 124 may include at least an aperture 123 (e.g., 123A or 123B) and a curved segment 125 (e.g., 125A or 125B). The inwardly projecting wall 124 may form a perimeter of an aperture 123A, 123B. For example, the apertures 123A, 123B and the respective curved segments 125A, 125B may define the boundaries, shape, configuration, and dimensions of the inwardly projecting wall 124.

The curved segments 125A, 125B may protrude inwardly into the working channel of the flexible section 120, forming a portion of the inwardly projecting wall 124. The position of the curved segments 125A, 125B may define the features of the apertures 123A, 123B. In an embodiment, the curved segments 125A, 125B may form a semi-circular shape and/or a U-shaped design, defining a similar or corresponding shape for the apertures 123A, 123B. For example, the apertures 123A, 123B may also include an opening of the inwardly projecting wall 124 forming a “U” shape and forming the perimeter of the inwardly projecting wall 124. The configuration of the curved segments 125A, 125B, the apertures 123A, 123B, and the perimeter of the inwardly projecting wall 124 is merely exemplary and may be adjusted based on the requirements of different medical procedures. For example, a medical procedure requiring the passage of control members 106 through the apertures 123A, 123B while restricting access to other components (e.g., Bowden cables 108 that abut a proximal surface of curved segments 125A, 125B.)

As shown in FIGS. 3A-3B, in an embodiment, there may be more than one inwardly projecting wall 124 within the distal portion 122 of the flexible section 120. The inwardly projecting walls 124 may be positioned on opposite sides of the inner surface 128 of the shaft 110. For example, there may be two inwardly projecting walls 124A, 124B, as labeled in FIG. 3B. The inwardly projecting walls 124A, 124B may include at least a curved segment 125A, 125B and two apertures 123A and 123B, each integrated within one of the inwardly projecting walls 124A, 124B. The curved segments 125A, 125B may each extend inward into the working channel of the flexible section 120 and define the shape of the apertures 123A and 123B.

As discussed above, the medical device 100 may include at least one control member(s) 106 extending from the proximal end 103 of the handle 102 to the distal end 114 of the shaft 110. The control member(s) 106 may attach (e.g., directly or indirectly) to the actuator 107 at the proximal end 103 of the handle 102. The actuator 107 may help to tension the control member(s) 106, and manipulation of the actuator 107 may help to actuate the articulation section 130.

The Bowden cable 108 may enclose or surround the control member(s) 106 from the proximal end 103 of the handle 102 to the proximal surface of the curved segments 125A, 125B. While surrounding the control member 106, the Bowden cable 108 may maintain a curved shape even when tensioned. The Bowden cable 108, therefore, may help to the control member 106 to move freely without becoming overly rigid, for example, while being tensioned by the actuator 107. As discussed below, the Bowden cable 108 may radially surround the control member 106 from the proximal end 103 of the handle 102 and terminate or abut the proximal surface of the plurality of the curved segments 125A, 125B (e.g., FIG. 3C).

As shown in FIG. 3A, the medical device 100 (FIG. 1) may be equipped with two control members 106, each positioned on opposing sides of the shaft 110, for example, on opposing sides of the working channel (not shown). Two Bowden cables 108 may at least partially surround each of the control members 106. In the embodiment, the control members 106 may extend through apertures 123A, 123B from to the distal end 114 of the shaft. The control member 106 may receive the inwardly projecting wall 124 within the flexible section 120 and extend through the apertures 123A, 123B described above. The size and/or shape of apertures 123A, 123B may be adjusted based on, for example, the dimension of control members 106. As shown in FIG. 3C, the two control members 106 may further extend through an aperture 132A defined by each articulation segment 132 of the articulation section 130.

The curved segment 125A, 125B may assist the Bowden cables 108 in remaining anchored within the proximal end 112 of the shaft 110 and terminate at a proximal surface of the respective curved segment (e.g., 125A, 125B). At the proximal end 103 of the handle 102, mechanisms or features may also be used to anchor the Bowden cable 108. Methods of anchoring may include using ferrules, crimps, or other mechanical fasteners that produce compression on the Bowden cable 108. The anchoring points of the Bowden cable 108 within the proximal end 103 of the handle 102 and on the curved segment 125A, 125B may help to ensure that the Bowden cables 108 remains in place while the control members 106 are being actuated and/or tensioned.

As discussed, the one or more control members 106 may extend through, for example, the apertures 123A, 123B. This extension may facilitate the connection between the flexible section 120 and the articulation section 130 while the Bowden cables 108 are seated on the proximal surface of the curved segment (e.g., 125a, 125b). Additionally, although only two control members 106 and two inwardly-projecting walls 124 (i.e., inwardly-projecting walls 124A, 124B are shown, it is noted that the medical device 100 may include any number of control members 106, Bowden cables 108, and inwardly-projecting walls 124. For example, if the shaft 110 is deflectable in four directions, then the medical device may include four control members 106, four Bowden cables 108, and four inwardly-projecting walls 124 (e.g., circumferentially spaced by approximately 90 degrees within the shaft and/or surrounding the working channel).

At least one of the one or more control members 106 may terminate at the end cap 136 (FIG. 2A) at the distal end 114 of the shaft 110. The end cap 136 may cover and enclose the distal end 114 of the shaft 110. The end cap 136 may provide a protective barrier for the control members 106. For example, the end cap 136 may help protect the control members 106 from damage or contamination during medical procedures.

Furthermore, the end cap 136 may incorporate various features. For example, the end cap 136 may integrate one or more lighting elements to illuminate the area of operation. Additionally, the end cap 136 could house a lens or imaging devices, allowing for imaging of the internal body structures. Moreover, in some configurations, the end cap may incorporate components related to the working channel, such as additional tools or sensors.

While principles of the disclosure are described herein with reference to illustrative aspects for particular medical uses and procedures, the disclosure is not limited thereto. Those having ordinary skill in the art and access to the teachings provided herein will recognize additional modifications, applications, aspects, and substitution of equivalents all fall in the scope of the aspects described herein. Accordingly, the disclosure is not to be considered as limited by the foregoing description.

What is claimed is:

1. A medical device comprising:
a shaft extending from a proximal end to a distal portion of the shaft, the distal portion of the shaft including:
a flexible section, wherein a distal portion of the flexible section includes at least one inwardly-projecting wall;
an articulation section including a plurality of articulation segments coupled together and rotatable relative to each other, a proximal-most articulation segment being attached to a distal end of the flexible section;
at least one control member extending from the proximal end of the shaft to the distal end of the shaft; and
at least one Bowden cable surrounding the at least one control member in the flexible section of the shaft, the at least one Bowden cable terminating at the at least one inwardly-projecting wall proximate the distal portion of the flexible section.

2. The medical device of claim 1, wherein the flexible section includes a tube that defines a plurality of slots.

3. The medical device of claim 2, wherein the distal portion of the flexible section that includes the at least one inwardly-projecting wall does not include the plurality of slots.

4. The medical device of claim 2, wherein the tube comprises a metal, a metal alloy, or a polymer.

5. The medical device of claim 2, wherein the at least one inwardly-projecting wall is an integral component of the tube.

6. The medical device of claim 2, wherein the at least one inwardly-projecting wall is coupled to an inner surface of the tube.

7. The medical device of claim 1, wherein the at least one control member includes two control members on opposite sides of the shaft, and wherein the at least one Bowden cable includes two Bowden cables each surrounding one of the two control members.

8. The medical device of claim 2, wherein the flexible section includes two inwardly-projecting walls on opposite sides of an inner surface of the shaft.

9. The medical device of claim 8, wherein at least two control members and at least two Bowden cables are positioned on opposite sides of the inner surface of the shaft.

10. The medical device of claim 1, wherein the at least one control member is received within the at least one inwardly-projecting wall.

11. The medical device of claim 1, wherein the at least one inwardly-projecting wall is a perimeter of an aperture, wherein the at least one control member extends through the aperture.

12. The medical device of claim 1, wherein the at least one control member extends through an aperture defined by each articulation segment of the articulation section.

13. The medical device of claim 1, wherein a distal-most articulation segment of the plurality of articulation segments is configured to be coupled to an end cap.

14. The medical device of claim 13, wherein the at least one control member extends through the distal-most articulation segment and is configured to terminate at the end cap.

15. The medical device of claim 1, further comprising a handle with an actuator operably coupled to the at least one control member, wherein manipulating the actuator tensions the at least one control member.

16. A medical device, comprising:
a handle;
a shaft comprising a flexible area and an articulation area, wherein the articulation area is configured to releasably couple to a flexible tube area, and wherein a distal portion of the flexible tube area includes a plurality of curved segments;
a plurality of pull wires extending distally from the handle through the shaft, wherein the plurality of pull wires connect the flexible tube area and the articulation area; and
a plurality of Bowden cables, wherein each of the plurality of Bowden cables radially surrounds at least a portion of one of the plurality of pull wires, wherein the plurality of Bowden cables abuts a proximal surface of the plurality of curved segments.

17. The medical device of claim 16, wherein each of the plurality of curved segments are formed by an at least one inwardly projecting wall of the flexible tube area and form an aperture.

18. The medical device of claim 17, wherein the plurality of Bowden cables abuts a proximal face of the at least one inwardly-projecting wall.

19. The medical device of claim 16, wherein the plurality of pull wires extend through an aperture defined by each articulation segment of the articulation area.

20. A medical device comprising:
a shaft extending from a proximal end to a distal end of the shaft, the distal end of the shaft including:
a flexible section, wherein a distal portion of the flexible section includes at least one inwardly-projecting wall;
an articulation section including a plurality of articulation segments;
at least one control member extending from the proximal end of the shaft to the distal end of the shaft, a proximal-most articulation segment being coupled to a distal end of the flexible section by the at least one control member; and
at least one Bowden cable surrounding at least a portion the at least one control member in the flexible section of the shaft, the at least one Bowden cable terminating at the at least one inwardly-projecting wall.
